(19) United States
(12) Patent Application Publication
(10) Pub. No.: US 2024/0350737 A1
(43) Pub. Date: Oct. 24, 2024

(54) SUB-ASSEMBLY OF A MEDICAMENT DELIVERY DEVICE

(71) Applicant: SHL MEDICAL AG, ZUG (CH)

(72) Inventors: Ming-Ting Yin, Taoyuan City (TW); Yen-Chun Chen, Taoyuan City (TW)

(21) Appl. No.: 18/565,243

(22) PCT Filed: May 24, 2022
(86) PCT No.: PCT/EP2022/064086

§ 371 Date: May 27, 2024

(30) Foreign Application Priority Data
Jun. 8, 2021 (EP) 21178288.3

(51) Int. Cl. A61M 5/315

(52) U.S. Cl. CPC A61M 5/31505; A61M 2207/10

(57) ABSTRACT

A sub-assembly of a medicament delivery device is disclosed having a first part containing a first fastener and a second fastener; a second part of the medicament delivery device sub-assembly having a first counter fastener and a second counter fastener; and a space between the first part and the second part; wherein the space is configured to accommodate a medicament container; wherein the medicament delivery device sub-assembly can have a shipping configuration where the distally directed surface of the first part abuts the proximally directed surface of the second part so that the first part and the second part are detachably attached to each other and an assembled configuration where the fastener of the first part abuts the counter fastener of the second part so that the first part and the second part are undetachably attached to each other.

### CROSS REFERENCE TO RELATED APPLICATIONS

The present application is a U.S. National Phase Application pursuant to 35 U.S.C. § 371 of International Application No. PCT/EP2022/064086 filed May 24, 2022, which claims priority to European Patent Application No. 21178288.3 filed Jun. 8, 2021. The entire disclosure contents of these applications are herewith incorporated by reference into the present application.

### TECHNICAL FIELD

The present disclosure generally relates to a sub-assembly of a medicament delivery device, especially a sub-assembly that has a shipping configuration and an assembled configuration.

### BACKGROUND

Medicament delivery devices such as pen type manual injectors or auto-injectors are generally known for the self-administration of a medicament by patients without formal medical training. For example, patients suffering from diabetes may require repeated injections of insulin, or patients may require regular injections of other types of medicaments, such as a growth hormone. For helping patients to correctly perform a self-administration, one or more indications for indicating one or more stages of an operation of a medicament delivery device is important.

A medicament delivery device comprises a primary package, namely a medicament and components that can directly contact the medicament, e.g. medicament container and medicament delivery member, and other operation components, namely components for constructing a medicament delivery device, that do not directly contact the medicament. Most of the time, the primary packages and the operation components will be manufactured in different locations. A location for selling medicament delivery devices to users will also often be different from the locations of manufacture of the primary package and/or the operation components. Therefore, the demand for shipping the primary packages, the operation components, and the medicament delivery devices is significant. However, requirements for shipping the primary packages and for shipping the operation components usually will be quite different, and the requirement for shipping the primary packages normally will be stricter than the requirement for shipping the operation components. Therefore, shipping both the operation components and the primary packages to the selling location (or the manufacture location of the primary packages, if it is closer to the location for selling) can be more efficient in comparison with shipping the primary packages to the manufacture location of the operation components, assembling them as medicament delivery devices, then shipping the medicament delivery devices a long way to the location for selling.

It has been appreciated that solutions for shipping the operation components as a plurality of sub-assemblies that can be simply assembled after a primary package has been inserted, thereby lowering the shipping cost and also reducing the complexity of the final step of assembling a medicament delivery device, could be advantageous.

### SUMMARY

The present disclosure is defined by the appended claims, to which reference should now be made.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which under use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which under use of the medicament delivery device is/are located closest to the dose delivery site.

Further, the term “longitudinal”, “longitudinally”, “axially” or “axial” refer to a direction extending from the proximal end to the distal end, typically along the device or components thereof in the direction of the longest extension of the device and/or component.

Similarly, the terms “transverse”, “transversal” and “transversally” refer to a direction generally perpendicular to the longitudinal direction.

Further, the terms “circumference”, “circumferential”, or “circumferentially” refer to a circumference or a circumferential direction relative to an axis, typically a central axis extending in the direction of the longest extension of the device and/or component. Similarly, “radial” or “radially” refer to a direction extending radially relative to the axis, and “rotation”, “rotational” and “rotationally” refer to rotation relative to the axis.

A first aspect of the present disclosure provides a sub-assembly of a medicament delivery device, the sub-assembly comprising: a first part comprising a first fastener and a second fastener; and a second part comprising a first counter fastener and a second counter fastener; the sub-assembly has a shipping configuration where the first fastener is releasably engaged with the first counter fastener; and an assembled configuration where the second fastener is engaged with the second counter fastener such that the first part and the second part are undetachably attached to each other.

Preferably, according to another embodiment, a friction-fit engagement is formed between the first fastener and the first counter fastener when the sub-assembly is in the shipping configuration.

Alternatively, according to another embodiment, the first fastener comprises a distally directed surface; and the first counter fastener comprises a proximally directed surface.

Preferably, according to another embodiment, the first fastener is arranged on a flexible portion of the first part and/or and the first counter fastener is arranged on a flexible portion of the second part.

Preferably, according to another embodiment, at least one of the proximally directed surface and the distally directed surface is angled relative to the longitudinal axis.

Preferably, according to another embodiment, the sub-assembly comprising a tubular housing for receiving a medicament container; wherein the tubular housing extends along a longitudinal axis between a proximal end and a distal end; and wherein the tubular housing is a part of one of the first part and the second part.

Preferably, according to another embodiment, the other one of the first part and the second part is at least partially arranged within the tubular housing and coaxial with the tubular housing.

Preferably, according to another embodiment, wherein the tubular housing comprises a ledge extending from an inner surface of a wall of the tubular housing.

Preferably, according to another embodiment, the proximally directed surface is a part of the ledge.

Preferably, according to another embodiment, the second counter fastener is a part of the ledge.

Preferably, according to another embodiment, a cut-out is arranged on a wall of the tubular housing.

Alternatively, according to another embodiment, the proximally directed surface is formed on an edge of the cut-out.

Alternatively, according to another embodiment, the second counter fastener is the cut-out.

Preferably, according to another embodiment, the tubular housing is a part of the second part; the first part comprises a distal cap; and the distal cap is configured to cover the distal end of the tubular housing of the second part when the sub-assembly is in the assembled configuration.

Preferably, according to another embodiment, the distal cap comprises a cover wall and a body wall; the cover wall extends in a direction transverse to the longitudinal axis; the body wall extends along the longitudinal axis from the cover wall in the proximal direction; and the body wall is at least partially arranged within the tubular housing.

Preferably, according to another embodiment, one of the second fastener and the counter second fastener comprises a second distally directed surface and the other one of the second fastener and the counter second fastener comprises a second proximal directed surface engaged with the second distally directed surface when the sub-assembly is in the assembled configuration.

Preferably, according to another embodiment, the second fastener comprises the second distally directed surface and the counter second fastener comprises the second proximal directed surface engaged with the second distally directed surface when the sub-assembly is in the assembled configuration.

Preferably, according to another embodiment, one of the second distally directed surface and the second proximally directed surface is angled relative to the longitudinal axis.

Preferably, according to another embodiment, the second distally directed surface of second fastener is the distally directed surface of the first fastener.

Preferably, according to another embodiment, the distal cap comprises a protrusion extending from an outer surface of the body wall.

Preferably, according to another embodiment, the second fastener is the protrusion extending from an outer surface of the body wall.

Preferably, according to another embodiment, the distally directed surface is a part of the protrusion.

Preferably, according to another embodiment, a cut-out is arranged on the body wall.

Alternatively, according to another embodiment, the second fastener is the cut-out arranged on the body wall.

Alternatively, according to another embodiment, the distally directed surface is a part of the cut-out arranged on the body wall.

Preferably, according to another embodiment, the first part comprises a rotator rotatably attached to the distal cap; and the rotator is rotatable relative to the distal cap around the longitudinal axis.

Preferably, according to another embodiment, the rotator is at least partially arranged within the tubular housing; and the rotator comprises a rotator body and a protrusion; the protrusion extends from an outer surface of the rotator body of the rotator; and the distally directed surface is a part of the protrusion.

Preferably, according to another embodiment, the first part comprises a slider axially movably attached to the distal cap of the first part; and the slider is axially movable relative to the distal cap along the longitudinal axis.

Preferably, according to another embodiment, the slider is arranged within the tubular housing; the slider comprises a slider body and a protrusion; the protrusion extends from an outer surface of the slider body; and the distally directed surface is a part of the protrusion.

Preferably, according to another embodiment, one of the first part and the second part comprises at least one of a medicament delivery member guard and a medicament container holder; and the medicament delivery member guard comprises an elongated portion extending along the longitudinal axis between the proximal end and the distal end.

Preferably, according to another embodiment, the medicament container holder comprises a tubular body extending along the longitudinal axis between the proximal end and the distal end.

Preferably, according to another embodiment, the at least one of the medicament delivery member guard and the medicament container holder is arranged within the tubular housing; the proximally directed surface is arranged on an inner surface or an outer surface of the medicament delivery member guard when the second part comprising the medicament delivery member guard; or the proximally directed surface is arranged on an inner surface or an outer surface of the medicament container holder when the second part comprising medicament container holder.

Preferably, according to another embodiment, the second counter fastener and the second fastener are circumferentially offset relative to each other around the longitudinal axis when the sub-assembly is in the shipping configuration.

Preferably, according to another embodiment, the second counter fastener and the second fastener are axially offset relative to each other along the longitudinal axis when the sub-assembly is in the shipping configuration.

Preferably, according to another embodiment, the second counter fastener and the second fastener are fastened to each other through a snap-fit joint when the sub-assembly is in the assembled configuration.

Preferably, according to another embodiment, when only the proximally directed surface is arranged on a flexible portion of the second part, the distally directed surface and a surface of the second fastener is the same surface; or when only the distally directed surface is arranged on a flexible portion of the first part, the distally directed surface and a surface of the second fastener is the same surface.

Alternatively, according to another embodiment, when only the proximally directed surface is arranged on a flexible portion of the second part, the proximally directed surface and a surface of the second counter fastener is the same surface; or when only the distally directed surface is arranged on a flexible portion of the first part, the distally directed surface and a surface of the second fastener is the same surface.

Preferably, according to another embodiment, the sub-assembly has a first measured dimension in the shipping configuration and a second measured dimension in the assembled configuration; and the first measured dimension is same as the second measured dimension.

Preferably, according to another embodiment, the first measured dimension and the second measured dimension are length as measured along a longitudinal axis.

Another aspect of the present disclosure provides a method of assembling a medicament delivery device comprising the sub-assembly comprising a first part comprising a first fastener and a second fastener; and a second part comprising a first counter fastener and a second counter fastener; and a tubular housing being a part of one of the first part and the second part; the sub-assembly has a shipping configuration where the first fastener is engage with the first counter fastener; and an assembled configuration where the second fastener is engaged with the second counter fastener, such that the first part and the second part are undetachably attached to each other, the method comprising the steps of: receiving the sub-assembly when the sub-assembly is in the shipping configuration; detaching the first part from the second part by an axial relative movement between the first part and the second part; inserting a medicament container into the tubular housing; and turning the sub-assembly into the assembled configuration by attaching the first part to the second part.

Preferably, according to another embodiment, the step of attaching the first part to the second part comprises fastening the first part to the second part.

Preferably, according to another embodiment, the step of turning the sub-assembly into the assembled configuration comprises rotating the first part relative to the second part before attaching the first part to the second part.

Preferably, according to another embodiment, the step of attaching the first part to the second part comprises axially moving the first part relative to the second part in the proximal direction until the second fastener engages with the second counter fastener.

Preferably, according to another embodiment, the step of inserting the medicament container into the tubular housing of the second part comprises inserting the medicament container from the distal end of the tubular housing of the second part.

Preferably, according to another embodiment, at least one of the first part and the second part comprise more than one component.

Preferably, according to another embodiment, at least one of the first part and the second part comprise more than one component.

Preferably, according to another embodiment, the medicament delivery device can be one of an injection device, an inhalation device, or a medical sprayer.

Preferably, according to another embodiment, the injection device can be an autoinjector, an on-body device, a safety syringe or a pen-type manual injector.

Another aspect of the present disclosure provides a system for shipping medicament delivery device sub-assemblies, the system comprising: a tray comprising a recess; wherein the recess extends along a longitudinal axis between a first end and a second end; and the tray comprises a protrusion extending from the recess in a direction transverse to the longitudinal axis; a first sub-assembly of the medicament delivery device positioned within the recess of the tray; and a second sub-assembly of the medicament delivery device positioned within the recess of the tray; the first sub-assembly extends from the first end of the recess to the protrusion of the recess; and the second sub-assembly extends from the second end of the recess to the protrusion of the recess.

Preferably, according to another embodiment, the first sub-assembly comprises a distal cap; the second sub-assembly comprises a tubular housing; the first sub-assembly is partially arranged within the tubular housing; and the protrusion is positioned between the distal cap of the first sub-assembly and the tubular housing of the second sub-assembly.

Generally, all terms used in the claims are to be interpreted according to their ordinary meaning in the technical field, unless explicitly defined otherwise herein. All references to “a/an/the element, apparatus, component, means, etc.” are to be interpreted openly as referring to at least one instance of the element, apparatus, component, means, etc., unless explicitly stated otherwise.

### BRIEF DESCRIPTION OF THE DRAWINGS

Embodiments of the inventive concept will now be described, by way of example only, with reference to the accompanying drawings, in which:

FIG. 1A schematically shows a perspective view of a first part of a sub-assembly of the present disclosure.

FIG. 1B schematically shows a perspective view of a second part of the sub-assembly of the present disclosure.

FIG. 1C schematically shows a perspective view of the first part of FIG. 1A with the second part of FIG. 1B.

FIG. 2A schematically shows a perspective view of the sub-assembly of FIGS. 1A-C in a shipping configuration.

FIG. 2B schematically shows a perspective view of the sub-assembly of FIGS. 1A-C in an assembled configuration.

FIG. 3 schematically shows an exploded view of the second part of FIG. 1B.

FIGS. 4A and 4B schematically show cross-section views of the sub-assembly of FIG. 2A.

FIGS. 5A and 5B schematically show cross-section views of the sub-assembly of FIG. 2B.

FIG. 6A schematically shows a perspective view of a first part of the sub-assembly of the present disclosure in another embodiment.

FIG. 6B schematically shows a perspective view of a second part of the sub-assembly of the present disclosure in another embodiment.

FIG. 6C schematically shows a perspective view of the first part of FIG. 6A with the second part of FIG. 6B.

FIG. 7A schematically shows a perspective view of the sub-assembly of FIGS. 6A-C in a shipping configuration.

FIG. 7B schematically shows a perspective view of the sub-assembly of FIGS. 6A-C in an assembled configuration.

FIG. 8 schematically shows an exploded view of the second part of FIG. 6B.

FIG. 9A schematically show cross-section views of a portion of the sub-assembly of FIG. 7A.

FIG. 9B schematically show perspective views of a portion of the sub-assembly of FIG. 7B.

FIG. 10A schematically shows a perspective view of the sub-assembly of the present disclosure in another embodiment.

FIG. 10B schematically shows a perspective view of the sub-assembly of FIG. 10A in a shipping configuration.

FIG. 10C schematically shows a perspective view of the sub-assembly of FIG. 10A in an assembled configuration.

FIG. 11 schematically shows a side view of a system for shipping medicament delivery device sub-assemblies.

### DETAILED DESCRIPTION

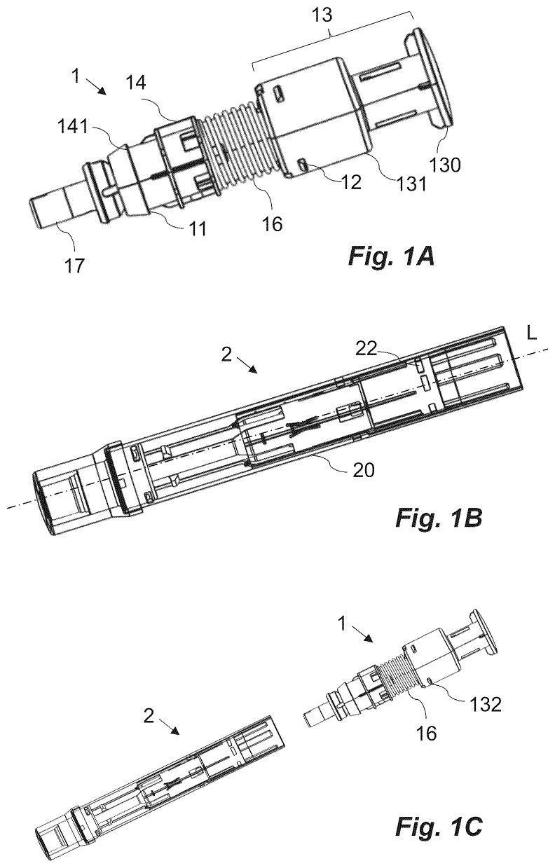

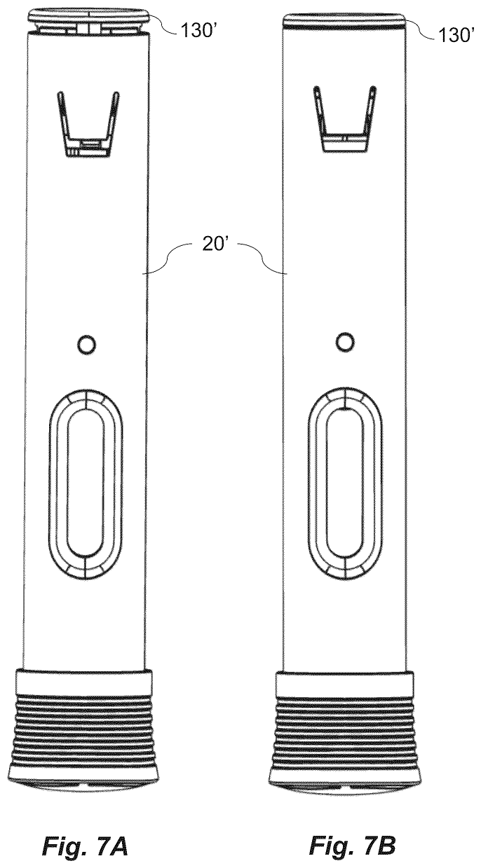

FIGS. 1-10C illustrate a first aspect of the present disclosure providing a sub-assembly of a medicament delivery device comprising a first part 1, 1′, 1″ and a second part 2, 2′, 2″. The first part 1, 1′, 1″ comprises a first fastener 11, 11′ and a second fastener 12, 12′. The second part 2, 2′, 2″ comprises a first counter fastener 21, 21′, 21″ and a second counter fastener 22, 22′, 22″.

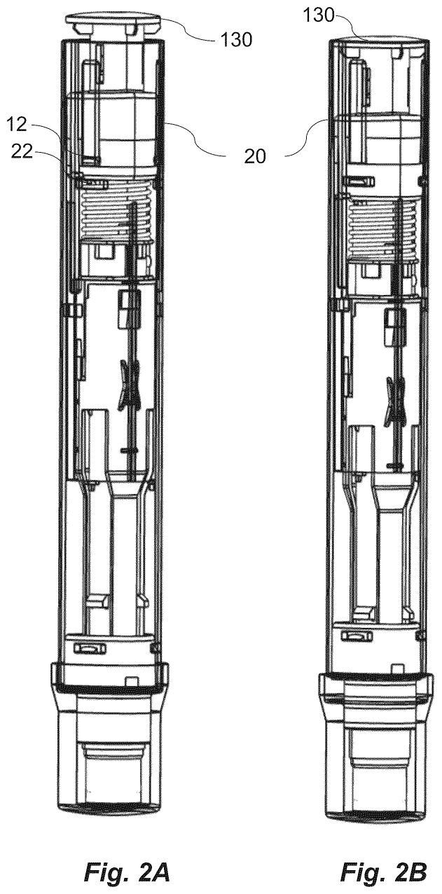

The sub-assembly has a shipping configuration where the first fastener 11, 11′ is engaged with the first counter fastener 21, 21′, 21″, as shown in FIGS. 2A, 7A, 10B; and an assembled configuration where the second fastener 12, 12′ is engaged with the second counter fastener 22, 22′, 22″, as shown in FIGS. 2B, 7B, 10C, so that the first part 1, 1′, 1″ and the second part 2, 2′, 2″ are undetachably attached to each other.

Therefore, when the sub-assembly is in the shipping configuration, an unintentional movement between the first part 1, 1′, 1″ and the second part 2, 2′, 2″ can be prevented. The sub-assembly can be thus delivered to and/or stored in a manufacture facility easily and more securely before a medicament container is inserted into the sub-assembly.

The engagement formed between the first fastener 11, 11; and the first counter fastener 21, 21′, 21″ when the sub-assembly is in the shipping configuration can be a friction-fit or a form-fit engagement.

In one example, the sub-assembly comprises a tubular housing 20, 20′, 10 for receiving a medicament container. The tubular housing 20, 20′, 10 extends along a longitudinal axis L between a proximal end and a distal end. The tubular housing 20, 20′, 10 is a part of one of the first part 1, 1′, 1″ and the second part 2, 2′, 2″; and the other one of the first part 1, 1′, 1″ and the second part 2, 2′, 2″ is at least partially arranged within the tubular housing 20, 20′, 10 and coaxially with the tubular housing 20, 20′, 10. For example, the tubular housing 20, 20′ is a part of the second part 2, 2′, and the first part 1, 1′, 1″ is partially coaxially relative to the longitudinal axis L arranged within the tubular housing 20, 20′ of the second part 2, 2′, 2″.

In one example, when the engagement formed between the first fastener 11, 11; and the first counter fastener 21, 21′, 21″ when the sub-assembly is in the shipping configuration is a form-fit engagement, the first fastener comprises a distally directed surface 11, 11′; and the first counter fastener comprises a proximally directed surface 21, 21′, 21″. In this example, when the sub-assembly is in the shipping configuration, the distally directed surface 11, 11′ is adjacent to the proximally directed surface 21, 21′. When the sub-assembly in the assembled configuration, the second fastener 12, 12′ is engaged with the second counter fastener 22, 22′, so that the first part and 1, 1′, 1″ the second part 2, 2′, 2″ are undetachably attached to each other; that is, once the sub-assembly is in the assembled configuration, the first part 1, 1′, 1″ and the second part 2, 2′, 2″ cannot be separated from each other without causing damage to the sub-assembly.

When the sub-assembly is in the shipping configuration, the sub-assembly may comprise all elements of a medicament delivery device except the medicament container, or except the medicament container and a medicament delivery member. Because the shipping cost for medicaments is relatively high, e.g. requirement of temperature control, being fragile, and higher value, shipping the sub-assembly without a medicament container and then assembling the sub-assembly with the medicament in a place that is close to the market can lower the cost of shipping. Simply assembling most of the components of the medicament delivery device as two parts and then temporarily attaching these two parts together for shipping can reduce the occupied space during shipping, and reduce the time consumption and complicity of a final assembling step (a step of generating a final, ready-for-the-use, product).

The abutment formed between the distally directed surface 11, 11′ and the proximally directed surface 21, 21′, 21″ can prevent the first part 1, 1′, 1″ being accidentally separated from the second part 2, 2′, 2″ during shipping the sub-assembly, e.g. if the sub-assembly is dropped or is shaken/hit during shipping.

Furthermore, because at least one of the distally directed surface 11, 11′ and the proximally directed surface 21, 21′ is on a flexible portion and at least one of the distally directed surface 11, 11′ and the proximally directed surface 21, 21′ is angled relative to the longitudinal axis L, e.g. 30-60 degrees, though this is dependent on the force required to detach the first part from the second part, the abutment formed between the distally directed surface 11, 11′ and the proximally directed surface 21, 21′ can be overcome with a high force that pulls the first part 1, 1′, 1″ relative to the second part 2, 2′, 2″ along the longitudinal axis L. Therefore, the first part 1, 1′, 1″ can be detached from the second part 2, 2′, 2″. The magnitude of the force for detaching the first part 1, 1′, 1″ from the second part 2, 2′, 2″ can vary depending on the particular design. The force feature can be set by certain factors, like the angle that the at least one of the distally directed surface and the proximally directed surface are angled relative to the longitudinal axis L, whether only one or both distally directed surface and the proximally directed surface should be angled relative to the longitudinal axis L, whether only one or both distally directed surface and the proximally directed surface should be arranged on the flexible portions.

Alternatively, when the sub-assembly is in the shipping configuration is a friction-fit, the first fastener does not comprise the distally directed surface, and the counter first fastener does not comprise the proximal directed surface. Instead, the first fastener comprises a surface facing in a direction transverse to the longitudinal axis L, and the counter fastener comprises a counter surface facing facing towards the surface of the first fastener. The counter surface is engaged with the surface in the shipping configuration of the sub-assembly. In one example, the surface of the first fastener is a rough surface, or is made of material that can generate high friction, e.g., rubber, so that when the counter surface of the counter first fastener is engaged with the surface in the shipping configuration of the sub-assembly, the friction between the surface and the counter surface prevent the first part being accidentally separated from the second part during shipping the sub-assembly, e.g. if the sub-assembly is dropped or is shaken/hit during shipping. Alternatively, both the surface of the first fastener and the counter surface of the counter first fastener are rough surfaces or are made of material that can generate high friction.

FIGS. 1A-5B illustrate an example of the sub-assembly of the first aspect of the present disclosure. In this example, the first part 1 of the sub-assembly is a power pack of the medicament delivery device. The first part 1 comprises a plunger rod 17, a power source (not shown), a slider 14 and a distal cap 13. The plunger rod 17 is configured to expel a contained medicament of the medicament delivery device, and the power source is configured to propel the plunger rod in the proximal direction. The power can come from any suitable power source such as a spring, a gas canister or a motor. The slider 14 is configured to initiate the proximal movement of the plunger rod 17 by an axial movement of the slider 14 relative to the distal cap 13. For example, the slider 14 can releasably hold the plunger rod 17 against a force from the power source, e.g. by engaging the plunger rod directly or interacting with another component that holds the plunger rod directly; or the axial movement of the slider 14 may be configured to trigger the power source to apply the force on the plunger rod, e.g. by switching on a switch of the motor or releasing a valve of the gas canister. The slider 14 can be axially moved by a component of the second part when the medicament delivery device has been assembled and is ready for use by a user.

In this example, as shown in FIGS. 1A-5B, the distal cap 13 comprises a cover wall 130 and a body wall 131. The cover wall 130 of the distal cap 13 extends in a direction transverse to the longitudinal axis L. The body wall 131 extends from the cover wall 130 in the proximal direction along the longitudinal axis L. The slider 14 comprises a slider body 140 and a protrusion 141 extending from an outer surface of the slider body 140. The slider 14 is axially movably attached to the distal cap 13, meaning that movement of the slider 14 will be limited so that the slider 14 can move axially relative to the distal cap 13 but cannot be fully spaced apart from the distal cap 13. For example, the slider 14 can be indirectly attached to the body wall 131 of the distal cap 13 via a bridge element between the slider 14 and the body wall 131, e.g. an extra tube or ring-shaped component dependent on the design, or the slider 14 can directly attach to the body wall. The slider 14 is axially movable relative to the body wall 131 of the distal cap 13 along the longitudinal axis L. In this example, the distally directed surface 11 is a part of the protrusion 141 of the slider 14; and the second fastener 12 is a protrusion protruding from an outer surface of the body wall 131 of the distal cap 13.

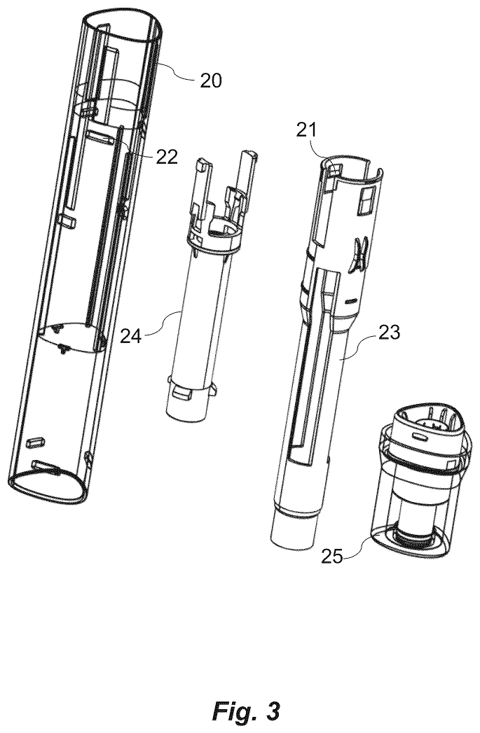

In this example, as shown in FIG. 3, the second part 2 of the sub-assembly comprises the tubular housing 20, a medicament delivery member guard 23, a medicament container holder 24, and a removable cap 25. The tubular housing 20 defines an interior space for receiving a medicament container. The medicament delivery member guard 23 and the medicament container holder 24 are arranged within the tubular housing 20. The removable cap 25 is attached to the proximal end of the tubular housing 20. The removable cap 25 is configured to seal a medicament delivery member from the environment before use of the medicament delivery device. The medicament delivery member guard 23 is configured to cover the medicament delivery member after the removable cap 25 has been removed. The medicament delivery member guard 23 is telescopic relative to the proximal end of the tubular housing 20. The medicament container holder 24 is configured to hold a medicament container directly. The medicament container holder 24 can be fixed to the tubular housing 20 or slidable relative to the tubular housing 20 along the longitudinal axis L. In this example, the proximally directed surface 21 is arranged on the flexible portion of the second part; preferably, the proximally directed surface 21 is arranged on a flexible tab of the medicament delivery member guard 23. In a preferred example, the flexible tab is arranged at the distal end of the medicament delivery member guard 23, as shown in FIG. 3.

In this example, the first part 1 is configured to be partly within the distal end of the tubular housing 20 of the second part 2 when the sub-assembly is in the shipping configuration and in the assembled configuration, as shown in FIG. 1C. In a preferred example, when the first part 1 is inserted into the distal end of the tubular housing 20, the slider 14 is also inserted into the tubular housing 20. The distal end of the tubular housing 20 is configured to be covered by the cover wall 130 of the distal cap 13 when the sub-assembly in the assembled configuration, as shown in FIG. 2B. In this example, the second counter fastener 22 is a cut-out arranged on a wall of the tubular housing 20, as shown in FIG. 3. Furthermore, in a preferred example, the first part 1 may comprise a medicament delivery member guard spring 16. As shown in FIGS. 1A and 1C, the medicament delivery member guard spring 16 is arranged between the distal end of the slider 14 and a ledge on the outer surface of the body wall of the distal cap 13. Therefore, when the sub-assembly in the assembled configuration, the medicament delivery member guard 23 of the second part 2 is telescopic relative to the proximal end of the tubular housing 20 under a force of the medicament delivery member guard spring 16 of the first part 1.

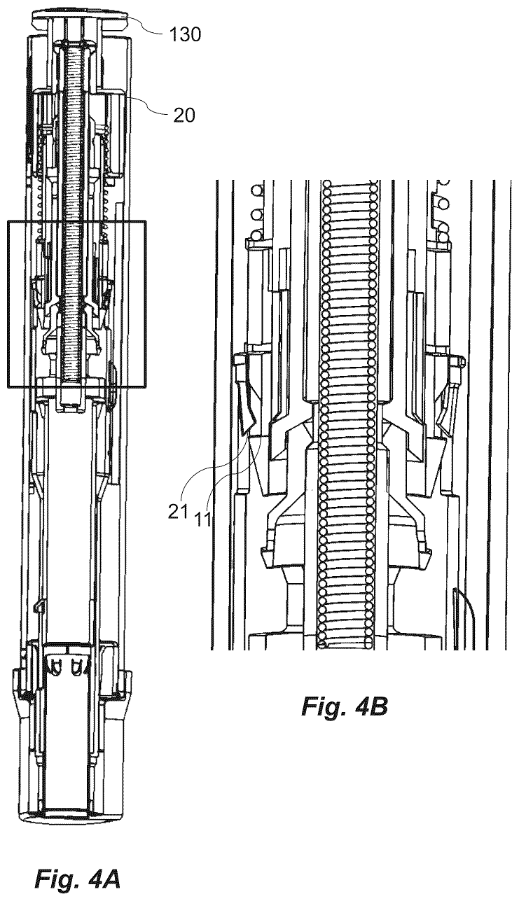

In this example, when the sub-assembly is in the shipping configuration, the distally directed surface 11 of the first part 1 is adjacent to the proximally directed surface 21 of the second part 2, as shown in FIGS. 4A-B. FIG. 4A shows the sub-assembly of this example in the shipping configuration, and FIG. 4B shows the highlighted portion of FIG. 4A. In this example, the proximally directed surface 21 is angled relative to the longitudinal axis L. When the first part 1 is pulled in the distal direction, the distally directed surface 11 will move along the angled, proximally directed surface 21 and deflect the flexible tab radially outward relative to the longitudinal axis L; therefore, the first part 1 can be detached from the second part 2. Therefore, for example, in a final assembling stage, the first part 1 can be detached from the second part 2, then a medicament container can be inserted into the tubular housing 20 of the second part 2 before putting the sub-assembly into the assembled configuration by attaching the first part 1 to the second part 2.

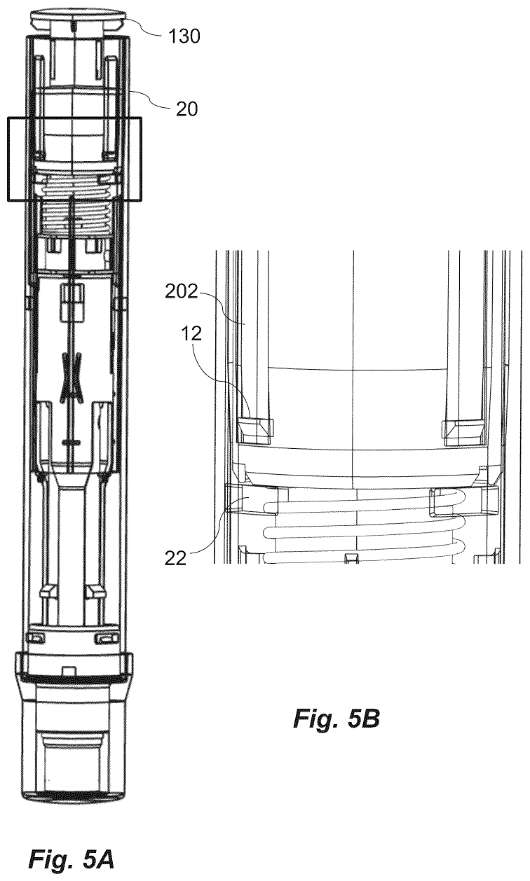

The second fastener 12 and the second counter fastener 22 can be either circumferentially offset relative to each other around the longitudinal axis L or axially offset relative to each other along the longitudinal axis L when the sub-assembly is in the shipping configuration. For example, when the second fastener and the second counter fastener are circumferentially offset relative to each other around the longitudinal axis L when the sub-assembly is in the shipping configuration, the tubular housing 20 of the second part 2 and/or the distal cap 13 of the first part 1 may comprise a structural arrangement for preventing the first part 1 from rotating relative to the second part 2, so that the sub-assembly will not be accidentally changed from the shipping configuration to the assembled configuration. The structural arrangement may be a pair of a slot and a rib, multiple pairs of ribs, or shapes of the distal cap and the tubular housing, e.g. the distal cap and the tubular housing can be triangular, square, or rectangular. In this example, the sub-assembly can be changed from the shipping configuration to the assembled configuration by pulling the first part in the distal direction until the first part detaches from the second part (a medicament container can be inserted into the tubular housing here or after the next step), rotating the first part around the longitudinal axis L relative to the second part, and inserting the first part partially into the tubular housing of the second part until the second fastener engages with the second counter fastener. It should be noted that rotating the first part relative to the second part can be done by actively rotating the first part relative to the second part; or actively rotating the second part relative to the first part; or actively rotating both the first part and the second part in opposite directions or with different rotational speeds. On the other hand, when the second fastener 12 and the second counter fastener 22 are axially offset relative to each other along the longitudinal axis L, the tubular housing 20 of the second part 2 and/or the distal cap 13 of the first part 1 may comprise a structural arrangement for temporarily preventing the first part 1 from axially moving relative to the second part 2 in the direction that may cause the second fastener 12 to engage with the second counter fastener 22, so that the sub-assembly will not be accidentally changed from the shipping configuration to the assembled configuration. For example, as shown in FIGS. 5A-B, the tubular housing 20 may comprise a slot 202 on an inner surface of the wall of the tubular housing 20. The second fastener 12 of the distal cap 13 is positioned in the slot 202 when the sub-assembly is in the shipping configuration. The second fastener 12 of the distal cap 13 comprises a distally directed surface angled relative to the longitudinal axis L. The second fastener 12 therefore can be prevented from accidentally moving into the cut-out 22 (the second counter fastener 22 in this example), e.g. by shaking, when the sub-assembly is in the shipping configuration. The sub-assembly can be turned into the assembled configuration by applying a significant axial force on the distal cap 14 until the second fastener 12 is engaged with the second counter fastener 22. In a preferred example, the cover wall contacts the distal end of the tubular housing 20 at the same time. It should be noted that the cover wall 130 of the distal cap 13 in this example has a dimension larger than the dimension of the distal end of the tubular housing 20 so that once the sub-assembly is in the assembled configuration, a further axial movement of the distal cap 13 in the proximal direction is blocked by the contact between the distal wall 130 of the distal cap 13 and the distal end of the tubular housing 20. Alternatively, the further axial movement of the distal cap 13 in the proximal direction can be blocked by the engagement of the second fastener and the second counter fastener, so that the distal wall 130 of the distal cap 13 can be dimensioned to snugly fit into the distal end of the tubular housing 20.

Furthermore, in one example, the sub-assembly has a first measured dimension in the shipping configuration and a second measured dimension in the assembled configuration. The first measured dimension is same as the second measured dimension. In a preferred example, the first measured dimension and the second measured dimension are length as measured along the longitudinal axis L. For example, as shown in FIGS. 2A-2B, upon moving the distal cap 13 in the proximal direction for the sub-assembly to the assembled configuration, components of the second part 2 except the tubular housing 20 are moved in the proximal direction. A length measured from the distal end of the tubular housing 20 to the proximal end of the distal wall 130 of the distal cap defines the moving distance of the components of the second part 2 except the tubular housing 20, so that the first measured length measured along the longitudinal axis L of the sub-assembly is same as the second measured length measured along the longitudinal axis L of the sub-assembly.

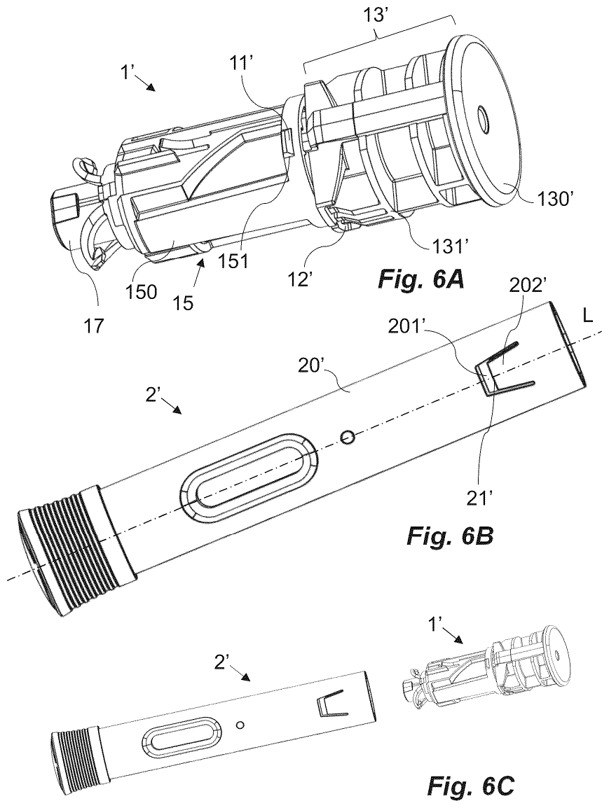

FIGS. 6A-9B illustrate another example of the sub-assembly of the first aspect of the present disclosure. In this example, the first part 1′ of the sub-assembly is a power pack of the medicament delivery device. The first part 1′ comprises a plunger rod 17, a power source (not shown), a rotator 15 and a distal cap 13′. The plunger rod 17 is configured to expel a contained medicament of the medicament delivery device, and the power source is configured to propel the plunger rod 17 in the proximal direction. The power can come from any suitable power source such as a spring, a gas canister or a motor. The rotator 15 is configured to initiate the proximal movement of the plunger rod 17 by a rotation of the rotator 15 relative to the distal cap 13′. For example, the rotator 15 can releasably hold the plunger rod 17 against a force from the power source, e.g. by engaging the plunger rod directly or interacting with another component that holds the plunger rod directly; or the rotation of the rotator 15 may be configured to trigger the power source to apply the force on the plunger rod, e.g. by switching on a switch of the motor or releasing a valve of the gas canister. The rotator 15 can be axially moved by a component of the second part when the medicament delivery device has been assembled and is ready for use by a user.

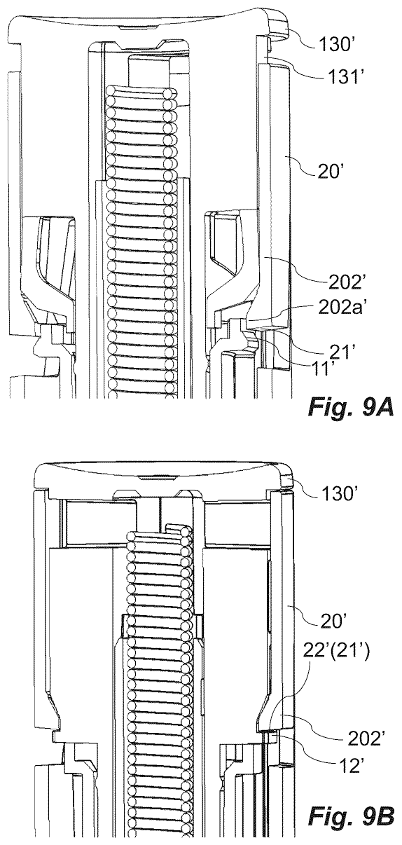

In the example, as shown in FIGS. 6A-9B, the distal cap 13′ comprises a cover wall 130′ and a body wall 131′. The cover wall 130′ of the distal cap 13′ extends in a direction transverse to the longitudinal axis L. The body wall 131′ extends from the cover wall 130′ in the proximal direction along the longitudinal axis L. The rotator 15 comprises a rotator body 150, and a protrusion 151 extending from an outer surface of the rotator body 150. The rotator 15 rotatably attaches to the distal cap 13′; for example, the rotator can be indirectly attached to the body wall 131′ of the distal cap 13′ via a conjunction element between the slider 14 and the body wall 131, or the rotator 15 can directly attach to the body wall 131′, so that the body wall 131′ may comprise an annular ledge extending from the body wall so that the rotator can rest on the annular ledge. The rotator 15 is rotatable relative to the body wall 131′ of the distal cap 13′ along the longitudinal axis L. In this example, the distally directed surface 11′ is a part of the protrusion 151 of the rotator 15; and the second fastener 12′ is a protrusion protruding from an outer surface of the body wall 131′ of the distal cap 13′. In this example, the distally directed surface 11′ is angled relative to the longitudinal axis L, as shown in FIG. 9A.

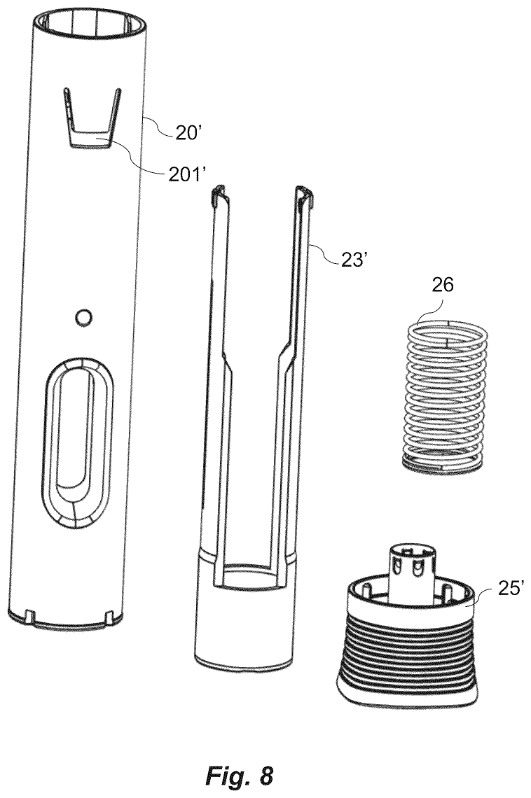

In this example, as shown in FIG. 8, the second part 2′ of the sub-assembly comprises the tubular housing 20′, a medicament delivery member guard 23′, a medicament delivery member guard spring 26, and a removable cap 25′. The tubular housing 20′ defines an interior space for receiving a medicament container. The medicament delivery member guard 23′ and the medicament delivery member guard spring 26 are arranged within the tubular housing 20′. The removable cap 25′ is attached to the proximal end of the tubular housing 20′. The removable cap 25′ is configured to seal a medicament delivery member from the environment before use of the medicament delivery device. The medicament delivery member guard 23′ is configured to cover the medicament delivery member after the removable cap 25′ has been removed. The medicament delivery member guard 23′ is telescopic relative to the proximal end of the tubular housing 20′ under a force from the medicament delivery member guard spring 26. In this example, a cut-out 201′ is arranged on a wall of the tubular housing 20′, and the wall of the tubular housing 20′ and the cut-out 201′ define a flexible tab 202′, as shown in FIGS. 6B and 9A-B. In this example, the proximally directed surface 21′ is arranged on the flexible portion of the second part 2′. In a preferred example, the proximally directed surface 21′ is arranged on an edge of the cut-out that is also a part of the flexible tab 202′. In a further preferred example, the flexible tab 202′ comprises an inward protrusion 202a′, as shown in FIG. 9A, extending from the flexible tab 202′ in the direction transverse to the longitudinal axis L toward the center of the tubular housing 20′. Therefore, the proximal directed surface 21′ is enlarged by the extension of the protrusion 202a′ of the flexible tab 202′ of the tubular housing 20′. In a preferred example, the flexible tab 202′ is arranged closer to the distal end of the tubular housing 20′ than to the proximal end of the tubular housing 20′, as shown in FIG. 9A.

In this example, the first part 1′ is configured to be partly within the distal end of the tubular housing 20′ of the second part 2′, when the sub-assembly is in the shipping configuration and in the assembled configuration, as shown in FIG. 6C. In a preferred example, when the first part 1′ is inserted into the distal end of the tubular housing 20′, the rotator 15 is also inserted into the tubular housing 20. The distal end of the tubular housing 20′ is configured to be covered by the cover wall 130′ of the distal cap 13′ when the sub-assembly in the assembled configuration, as shown in FIG. 5B. In this example, the cut-out 201′ is the second counter fastener 22′, namely, the proximally directed surface 21′ is the same as a surface of the second counter fastener 22′, as shown in FIGS. 9A-B.

In this example, when the sub-assembly is in the shipping configuration, the distally directed surface 11′ of the first part 1′ is adjacent to the proximally directed surface 21′ of the second part 2′, as shown in FIG. 9A. In this example, the distally directed surface 11′ is angled relative to the longitudinal axis L. When the first part 1′ is pulled in the distal direction, the angled, distally directed surface 11′ will deflect the flexible tab 202′ radially outward relative to the longitudinal axis L; therefore, the first part 1′ can be detached from the second part 2′.

The second fastener 12′ and the second counter fastener 22′ can be either circumferentially offset relative to each other around the longitudinal axis L or axially offset relative to each other when the sub-assembly is in the shipping configuration as mentioned above, and a structural arrangement for preventing the sub-assembly from being accidentally changed from the shipping configuration to the assembled configuration can be introduced into the design of the sub-assembly as well. For example, as shown in FIG. 6A, the second fastener 12′ is circumferentially offset relative to the distally directed surface 11′ around the longitudinal axis L by 90 degrees (alternatively, this angle could be smaller or larger, e.g. between 15 and 180 degrees, between 30 and 150 degrees, or between 60 and 120 degrees). Therefore, the sub-assembly can be changed from the shipping configuration to the assembled configuration by pulling the first part 1′ in the distal direction until the first part 1′ detaches from the second part 2′, rotating the first part 1′ around the longitudinal axis L relative to the second part 2′, and inserting the first part partially into the tubular housing 20′ of the second part 2′ until the second fastener 12′ is adjacent to the second counter fastener 22′, as shown in FIG. 9B. This arrangement also has the benefit that the proximally directed surface 21′ and the second counter fastener 22′ can be formed by sharing the same portion of the second part 2′, the manufacturing complexity of the sub-assembly can thus be reduced.

It should be noted that the cover wall 130′ of the distal cap 13′ in this example, may have a dimension larger than the dimension of the distal end of the tubular housing 20′, so that once the sub-assembly is in the assembled configuration, a further axial movement of the distal cap 13′ in the proximal direction will be blocked by the contact between the distal wall 130′ of the distal cap 13′ and the distal end of the tubular housing 20′. Alternatively, the further axial movement of the distal cap 13′ in the proximal direction can be blocked by the engagement of the second fastener and the second counter fastener, so that the distal wall 130′ of the distal cap 13′ can be dimensioned to snugly fit into the distal end of the tubular housing 20′.

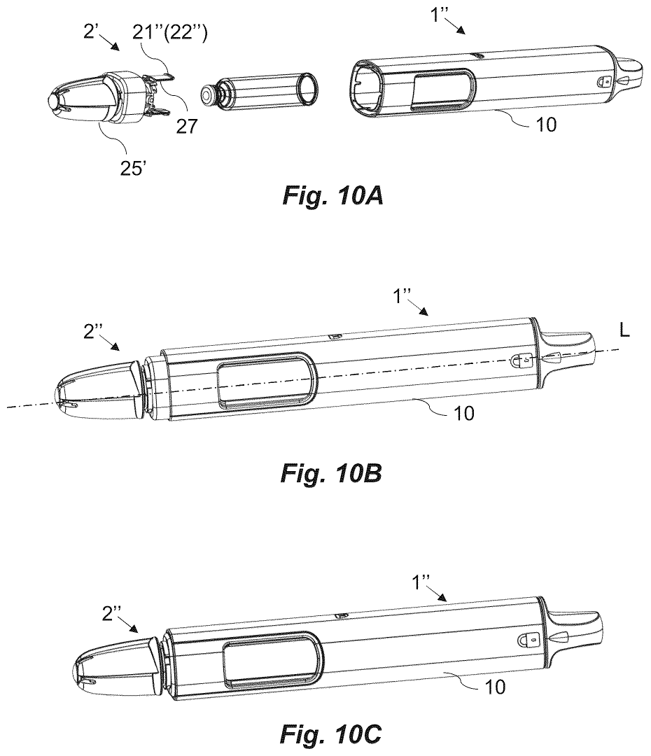

It should be noted that the first part 1″ can comprise a tubular housing 10 for containing a medicament container, as shown in FIGS. 10A-C. In this example, the distally directed surface and the second fastener can be arranged on an inner surface of a wall of the tubular housing 10. For example, the tubular housing 10 may comprise a ledge extending from the inner surface of the wall of the tubular housing 10; and the distally directed surface is a part of the ledge; and the second fastener can be a cut-out or a recess on the wall of the housing. In this example, the proximally directed surface 21″ and a surface of the second counter fastener 22″ may be the same on the second part 2″, for example, the second part 2″ may comprise a cap assembly with a cap portion 25″ and a flexible arm 27 extending from the cap portion in the distal direction along the longitudinal axis L. The flexible arm 27 may comprise a protrusion extending in the direction transverse to the longitudinal axis and define the proximally directed surface 21″ and the second counter fastener 22″.

The first part 1, 1′, 1″ and the second part 2, 2′, 2″are fastened to each other when the sub-assembly is in the assembled configuration. As illustrated in FIGS. 1A-9B, the engagement between the second fastener 12, 12, 12″ and the first counter fastener 21, 21′, 21″ can be a snap-fit joint, so that the first part 1, 1′, 1″ and the second part 2, 2′, 2″ are undetachable relative to each other. It should be noted that, alternatively, the second fastener can also be a form-fit or frictional form-fit joint or any other suitable fastening joint.

For assembling a medicament delivery device comprising a sub-assembly as mentioned, the assembly steps can be done by receiving the sub-assembly when the sub-assembly is in the shipping configuration; detaching the first part 1, 1′, 1″ from the second part 2, 2′, 2″ by an axial relative movement between the first part 1, 1′, 1″ and the second part 2, 2′, 2″; inserting a medicament container into the tubular housing 20, 20′, 10 of one of the first part 1, 1′, 1″ and the second part 2, 2′, 2″; and turning the sub-assembly into the assembled configuration by attaching the first part 1, 1′, 1″ to the second part 2, 2′, 2″. When the second fastener 12, 12′ and the second counter fastener 22, 22′, 22″ are axially offset relative to each other when the sub-assembly is in the shipping configuration, the step of turning the sub-assembly into the assembled configuration may be just simply moving the first part 1, 1′, 1″ to the second part 2, 2′, 2″ until the second fastener 12, 12′ and the second counter fastener 22, 22′, 22″engage each other. On the other hand, when the second fastener 12, 12′ and the second counter fastener 22, 22′, 22″ are angularly offset relative to the longitudinal axis from each other when the sub-assembly is in the shipping configuration, the step of turning the sub-assembly into the assembled configuration should be rotating the first part relative to the second part before attaching the first part to the second part. The medicament container can be inserted into the tubular housing 20, 20′ from the distal end of the tubular housing 20, 20′ as with the examples of the sub-assembly shown in FIGS. 1C-9B. Alternatively, The medicament container can be inserted into the tubular housing 10 from the proximal end of the tubular housing 10 as in the example of the sub-assembly shown in FIGS. 10A-C.

Furthermore, in the sub-assembly as mentioned above, when the second fastener 12, 12′ and the second counter fastener 22, 22′, 22″ are angularly offset relative to the longitudinal axis L related to each other, one of the first part 1, 1′, 1″ and the second part 2, 2′, 2″ can be either partially or fully inserted into the tubular housing 20, 20′ 10 of the other one of the first part 1, 1′, 1″ and the second part 2, 2′, 2″ when the sub-assembly is in the shipping configuration. Partially or fully inserting one of the first part 1, 1′, 1″ and the second part 2, 2′, 2′' into the tubular housing 20, 20′ 10 of the other one of the first part 1, 1′, 1″ and the second part 2, 2′, 2″ when the sub-assembly is in the shipping configuration can provide the advantage of reducing the space for shipping, and therefore reducing the cost of shipping.

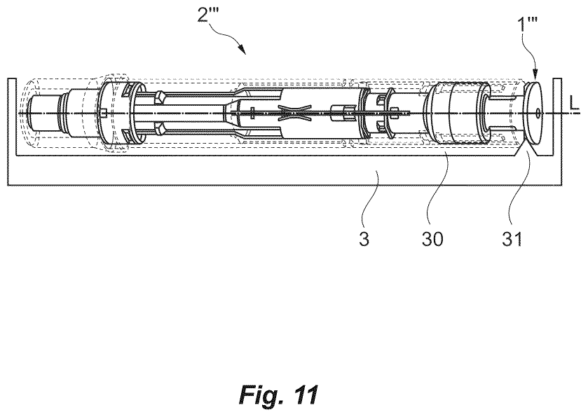

FIG. 11 illustrates the second aspect of the present disclosure and provides a system for shipping medicament delivery device sub-assemblies. The system comprises a tray 3, a first sub-assembly of the medicament delivery device 1″′ and a second sub-assembly of the medicament delivery device 2″′. The tray 3 comprises a recess 30 extending along a longitudinal axis between a first end and a second end; and a protrusion 31 extending from the recess 30 in a direction transverse to the longitudinal axis L. The first sub-assembly 1″′ of the medicament delivery device is configured to be positioned within the recess 30 of the tray 3. The first sub-assembly 1″′ extends from the first end of the recess to the protrusion 31 of the recess 30. The second sub-assembly 2″′ of the medicament delivery device is configured to be positioned within the recess 30 of the tray 3. The second sub-assembly 2″′ extends from the second end of the recess to the protrusion 31 of the recess 30. Therefore, when the first and second sub-assemblies of the medicament delivery device can be prevented from falling apart or accidentally undetachably attaching to each other during the shipping. In a preferred example, similar to the examples mentioned above, the first sub-assembly 1″′ comprises a distal cap 13″′ and the second sub-assembly 2″′ comprises a tubular housing 20″′. The first sub-assembly 1″′ is partially arranged within the tubular housing 20″′; and the protrusion 31 is positioned between the distal cap 14″′ of the first sub-assembly 1″′ and the tubular housing 20″′ of the second sub-assembly 2″′.

The inventive concept has mainly been described above with reference to a few examples. However, as is readily appreciated by a person skilled in the art, other embodiments than the ones disclosed above are equally possible within the scope of the inventive concept, as defined by the appended claims.

1-15. (canceled)

16. A sub-assembly of a medicament delivery device, the sub-assembly comprising:
a first part comprising a first fastener and a second fastener; and
a second part comprising a first counter fastener and a second counter fastener;
wherein the sub-assembly has
a shipping configuration where the first fastener is releasably engaged with the first counter fastener; and
an assembled configuration where the second fastener is engaged with the second counter fastener such that the first part and the second part are undetachably attached to each other.

17. The sub-assembly according to claim 16, wherein the first fastener comprises a distally directed surface; and wherein the first counter fastener comprises a proximally directed surface.

18. The sub-assembly according to claim 17, wherein the first fastener is arranged on a flexible portion of the first part and/or and the first counter fastener is arranged on a flexible portion of the second part.

19. The sub-assembly according to claim 17, wherein at least one of the proximally directed surface and the distally directed surface is angled relative to the longitudinal axis (L).

20. The sub-assembly according to claim 16, the sub-assembly comprising a tubular housing for receiving a medicament container; wherein the tubular housing extends along a longitudinal axis between a proximal end and a distal end; and wherein the tubular housing is a part of one of the first part and the second part.

21. The sub-assembly according to claim 20, wherein the other one of the first part and the second part is at least partially arranged within the tubular housing and coaxial with the tubular housing.

22. The sub-assembly according to claims 20, wherein the tubular housing is a part of the second part; wherein the first part comprises a distal cap; and wherein the distal cap is configured to cover the distal end of the tubular housing of the second part when the sub-assembly is in the assembled configuration.

23. The sub-assembly according to claim 22, wherein the first part comprises a rotator rotatably attached to the distal cap; wherein the rotator is rotatable relative to the distal cap around the longitudinal axis; wherein the rotator is at least partially arranged within the tubular housing; and wherein the rotator comprises a rotator body and a protrusion; wherein the protrusion extends from an outer surface of the rotator body of the rotator; and wherein the distally directed surface is a part of the protrusion.

24. The sub-assembly according to claim 22, wherein the first part comprises a slider axially movably attached to the distal cap of the first part; wherein the slider is axially movable relative to the distal cap along the longitudinal axis; wherein the slider is arranged within the tubular housing; wherein the slider comprises a slider body and a protrusion; wherein the protrusion extends from an outer surface of the slider body; and wherein the distally directed surface is a part of the protrusion.

25. The sub-assembly according to claim 16, wherein the second counter fastener and the second fastener are circumferentially offset relative to each other around the longitudinal axis when the sub-assembly is in the shipping configuration.

26. The sub-assembly according to claim 16, wherein the second counter fastener and the second fastener are axially offset relative to each other along the longitudinal axis when the sub-assembly is in the shipping configuration.

27. The sub-assembly according to claim 18, wherein when only the proximally directed surface is arranged on a flexible portion of the second part, the proximally directed surface and a surface of the second counter fastener is the same surface; or when only the distally directed surface is arranged on a flexible portion of the first part, the distally directed surface and a surface of the second fastener is the same surface.

28. A method of assembling a medicament delivery device comprising a sub-assembly, wherein the sub-assembly comprises a first part comprising a first fastener and a second fastener; and
a second part comprising a first counter fastener and a second counter fastener; and a tubular housing being a part of one of the first part and the second part; wherein the sub-assembly has a shipping configuration where the first fastener is engage with the first counter fastener; and an assembled configuration where the second fastener is engaged with the second counter fastener, such that the first part and the second part are undetachably attached to each other, the method comprising the steps of:
receiving the sub-assembly when the sub-assembly is in the shipping configuration;
detaching the first part from the second part by an axial relative movement between the first part and the second part;
inserting a medicament container into the tubular housing; and
turning the sub-assembly into the assembled configuration by attaching the first part to the second part.

29. The method of assembling a medicament delivery device according to method claim 28, wherein the step of turning the sub-assembly into the assembled configuration comprises rotating the first part relative to the second part before attaching the first part to the second part.

30. The method of assembling a medicament delivery device according to claim 28, wherein the step of attaching the first part to the second part comprises axially moving the first part relative to the second part in the proximal direction until the second fastener engages with the second counter fastener.

31. A sub-assembly of a medicament delivery device, the sub-assembly comprising:
a first part comprising a first fastener and a second fastener; and
a second part comprising a first counter fastener and a second counter fastener,
wherein axial relative movement between the first part and the second part causes the sub-assembly to change from a shipping configuration to a disassembled configuration and then to an assembled configuration, and
wherein when the sub-assembly is in the shipping configuration, the first fastener is releasably engaged with the first counter fastener, and the second fastener is not engaged with the second counter fastener.

32. The sub-assembly of claim 31, wherein when the sub-assembly is in the assembled configuration, the second fastener is engaged with the second counter fastener such that the first part and the second part are undetachably attached to each other.

33. The sub-assembly of claim 31, wherein when the sub-assembly is in the disassembled configuration, the first fastener is not engaged with the first counter fastener and the second fastener is not engaged with the second counter fastener.

34. The sub-assembly of claim 31, wherein relative rotational and axial movement between the first part and the second part causes the sub-assembly to change from the disassembled configuration to the assembled configuration.

35. The sub-assembly of claim 31, wherein the second counter fastener and the second fastener are axially offset relative to each other along the longitudinal axis when the sub-assembly is in the shipping configuration.
